(19) United States
(12) Patent Application Publication
(10) Pub. No.: US 2015/0174059 A1
(43) Pub. Date: Jun. 25, 2015

(54) ORAL CARE

(71) Applicant: University of Pretoria, Hillcrest (ZA)

(72) Inventors: Cynthia Joan HENLEY-SMITH, Muckleneuk (ZA); Namrita LALL, Hillcrest (ZA); Francina Susanna BOTHA, Brummeria (ZA); Ahmed Abdel-fattah HUSSEIN, Belleville (ZA)

(73) Assignee: UNIVERSITY OF PRETORIA, Hillcrest (ZA)

(21) Appl. No.: 14/632,683

(22) Filed: Feb. 26, 2015

Related U.S. Application Data
(63) Continuation of application No. PCT/IB2013/058016, filed on Aug. 27, 2013.

(30) Foreign Application Priority Data
Aug. 27, 2012 (ZA) 2012/01415

(51) Int. Cl. A61K 8/97; A61Q 11/00; C12Q 1/02; A61K 8/35

(52) U.S. Cl. CPC A61K 8/97; A61K 8/35; A61Q 11/00; C12Q 1/02; A61K 2800/59

(57) ABSTRACT

Various disclosed embodiments relate to oral care are presented. In particular, one embodiments includes to an oral care composition for inhibiting the growth of potentially pathogenic oral microorganisms, said composition comprising an extract of Heteropyxis natalensis. Another embodiment further includes an oral care composition which comprises Heteropyxis natalensis in combination with other essential oils and plant extracts.

THIS disclosure relates to oral care. In particular the invention relates to an oral care composition, the use of an extract of Heteropyxis natalensis for inhibiting the growth of potentially pathogenic oral microorganisms, use of a new composition, a substance or composition for use in a method of treating periodontal disease, a new use of Aurentiacin A or derivatives thereof, and a new method of evaluating the attachment of microorganisms to an enamel surface of a tooth and the effect of a composition on such attachment.

### BACKGROUND

Biofilms (plaque) are formed from the extensive growth of microorganisms, resulting from changes in the oral bacterial ecosystem. Once a biofilm is established it may lead to the formation of dental caries (tooth decay) or even more severe periodontal diseases. Dental caries and periodontal diseases in humans have an astonishing impact on the health and welfare of communities (Samaranayake, 2002). Sick leave, due to oral infections, and the consequent cost of dental treatment results in costing billions of dollars each year (Samaranayake, 2002). In 2007, the World Health Organization (WHO) stated that 5-10% of public health expenditure was related to dental care. Tooth decay and, to a lesser extent periodontal infections, are perhaps the most expensive infections that most individuals have to contend with, during a lifetime (Loesche, 1986). Natural plant products are becoming increasingly popular treatments, even for oral health care.

One of the fastest growing sectors in the agribusiness industry is the natural plant products which led to worldwide sales of $23 billion in 2002 alone. In 2008, there were approximately 85,000 medicinally useful plant species; however Africa only contributed 1% to the market even though 75% of the African population still relies on traditional herbal medicine (Makunga et al., 2008).

The inventor evaluated an indigenous plant from South Africa, Heteropyxis natalensis for bioactivity against pathogens which cause oral infections and oral diseases in humans. Identification of the bioactive principles from Heteropyxis natalensis has also been attempted.

The rationale of this study was to determine the antimicrobial activity of H. natalensis against four pathogenic microorganisms, Actinomyces israelii, Streptococcus mutans, Prevotella intermedia and Candida albicans. The synergistic effect of the combination of H. natalensis with the essential oils Melaleuca alternifolia (Tea tree), Mentha piperita (peppermint) and a concentrated green tea extract was investigated. As well as determine bacterial adhesion in the presence of H. natalensis. During the study flavonoids were isolated using bioassay guided fractionation.

The various disclosed embodiments aim to address the need for a natural product, which can prevent the colonization of potentially pathogenic oral bacteria in the mouth.

In this specification reference is made to the following documents:
Alviano, W. S., Alviano, D. S., Diniz, C. G., Antoniolli, A. R., Alviano, C. S., Farias, L. M., Carvalho, M. A. R., Souza, M. M. G., Bolognese, A. M. 2008. In vitro antioxidant potential of medicinal plant extracts and their activities against oral bacteria based on Brazilian folk medicine. Archives of Oral Biology, 53(6): 545-552.
Basson, A. E. 2005. Cell Culture Manual.
Braithwaite, M., van Vuuren, S. F., Viljoen, A. M. 2008. Validation of smoke inhalation therapy to treat microbial infections. Journal of Ethnopharmacology, 119: 501-506.
Cohen, M. A., Husband, M. D., Yoder, S. L., Gage, J. W., Roland, G. E. 1998. Bacterial eradication by clinafloxacin, CI-990, and ciprofloxacin employing MBC test, in-vitro time kill and in-vivo time-kill studies. Journal of Antimicrobial Chemotherapy, 41: 605-614.
Dictionary of Natural Products, version 20.1, 2011. Chapman and Hall, London.
Dominguez, X. A., Franco, R., Zamudio, A., Barradas, D. M., Watson, W. H., Zabel, V., Merijanian, A. 1980. Flavonoids from Dalea scandens var. paucifolia and Dalea thyrsiflora. Phytochemisty, 19: 1262-1263.
Du Toit, R., Volsteedt, Y., Apostolides, Z. 2001. Comparison of the antioxidant content of fruits, vegetables and teas measured as vitamin C equivalents. Toxicology, 166: 63-69.
Eloff, J. N. 1998. A sensitive and quick microplate method to determine the minimal inhibitory concentration of plant extract for bacteria. Plant Medica, 64: 711-713.
Fang, J., Paetz, C., Schneider, B. 2011. C-methylated flavonoids and dihydrochalcones from Myrica gale seeds. Biochemical Systematics and Ecology, 39(1): 68-70.
Fucundo, V. A. and Braz-Filho, R. 2004. C-methylated flavonoids from the roots of Piper carniconnectivum C. DC. (Piperaceae). Biochemical Systematics and Ecology, 32: 1215-1217.
Gafner, S., Wolfender, J-L., Mavi, S., Hostettmann, K. 1996. Abstract. Antifungal and antibacterial chalcones from Myrica serrate. Planta Medica, 62(1): 67-69.
Glauert, A. M. 1975. Fixation, dehydration and embedding of biological specimens in: Practical methods in electron microscopy. North-Holland Publishing, Amsterdam.
Hayat, M. A. 1981. Principles and techniques of electron microscopy, biological applications. 1 University Park Press, Baltimore.
Geoghegan, F., Wong, R. W. K., Rabie, A. B. M. 2010. Inhibitory effect of quercetin on periodontal pathogens in vitro. Phytotherapy Research, 24: 817-820.
Gundidza, M., Deans, S. G., Kennedy, A. I., Mavi, S., Waterman, P. G., Gray, A. I. 1993. The essential oil from Heteropyxis natalensis Harv: Its antimicrobial activities and phytocontituents. Journal of the Science of Food and Agriculture, 63: 361-364.
Hsieh, Y-L., Fang, J-M., Cheng, Y-S. 1997. Terpenoids and flavonoids from Pseudotsuga wilsoniana. Phytochemistry, 47(5): 845-850.
Mayer, R. 1989. Flavonoids from Leptospermum scoparium. Phytochemistry, 29 (4): 1340-1342.
McFarland, J. 1907. The nephelometer: An instrument for estimating the number of bacteria in suspensions for calculating the opsonic index and for vaccines. Journal of America Medical Association, 49: 1176.
Muanda, F. N., Soulimani, R., Dicko, A. 2011. Study on biological activities and chemical composition of extracts from Desmodium adscendens leaves. Journal of Natural Products, 4: 100-107.
Muchuweti, M., Nyamukonda, L., Chagonda, L. S., Ndhlala, A. R., Mupure, C., Benhura, M. 2006.
Total phenolic content and antioxidant activity in selected medicinal plants of Zimbabwe. International Journal of Food Science and Technology, 41: 33-38.
Mustafa, K. A., Kjaergaard, H. G., Perry, N. B., Weavers, R. T. 2003. Hydrogen-bonded rotamers of 2′,4′,6′-trihydroxy-3′-formyldihydrochalcone, an intermediate in the synthesis of a dihydrochalcone from Leptospermum recurvum. Tetrahedron, 59: 6113-6120.
Song, J. M. and Seong, B. L. 2007. Tea catechins as a potential altenative anti-infectious agent. Expert Review of Anti-infective Therapy, 5(3): 497-506.
Van Vuuren, S. F., Viljoen, A. M., Özek, T., Demirci, B., Baser, K. H. C. 2007. Seasonal and geographical variation of Heteropyxis natalensis essential oil and the effect thereof on the antimicrobial activity. South African Journal of Botany, 73: 441-448.
Van Wyk, B. and Gericke, N. 2000. General medicines, Chapter 7. Dental care, Chapter 12. Perfumes and repellents, Chapter 13. In: People's plants. Briza Publications, Pretoria, South Africa, pp. 119-228.
Van Wyk, B. and van Wyk, P. 1997. Field guide to trees of Southern Africa. Struik Publishers, South Africa, pp. 198-500.
Vilela, R. M., Lands, L. C., Meehan, B., Kubow, S. 2006. Inhibition of IL-8 release from CFTR-deficient lung epithelial cells following pre-treatment with fenretinide. International Immunopharmacology, 6: 1651-1664.

### SUMMARY

The disclosure relates to an oral care composition for inhibiting the growth of potentially pathogenic oral microorganisms, said composition comprising an extract of Heteropyxis natalensis.

The pathogenic oral microorganisms may include any one or more microorganisms selected from the group of Actinomyces israelli, Streptococcus mutans, Prevotella intermedia and Candida albicans.

In one embodiment in which the pathogenic oral microorganism is Actinomyces israelli, the concentration of the extract in the oral care composition may be at least 0.88 mg/ml.

In another embodiment in which the pathogenic oral microorganism is Streptococcus mutans, the concentration of the extract in the oral care composition may be at least 1.82 mg/ml. In such embodiment the composition may interfere with pellicle formation and glucan binding of S. mutans to an enamel surface of a tooth.

In a further embodiment in which the pathogenic oral microorganism is Prevotella intermedia, the concentration of the extract in the oral care composition may be at least 3.13 mg/ml.

In an embodiment in which the pathogenic oral microorganism is Candida albicans, the concentration of the extract in the oral care composition may be at least 8.33 mg/ml.

The extract of Heteropyxis natalensis may be in the form of an alcohol extract. More particularly the extract may be an ethanol extract. In another embodiment the extract may be a water extract.

The extract may include or be enriched for any one or more of the compounds selected from the group having the following structures:

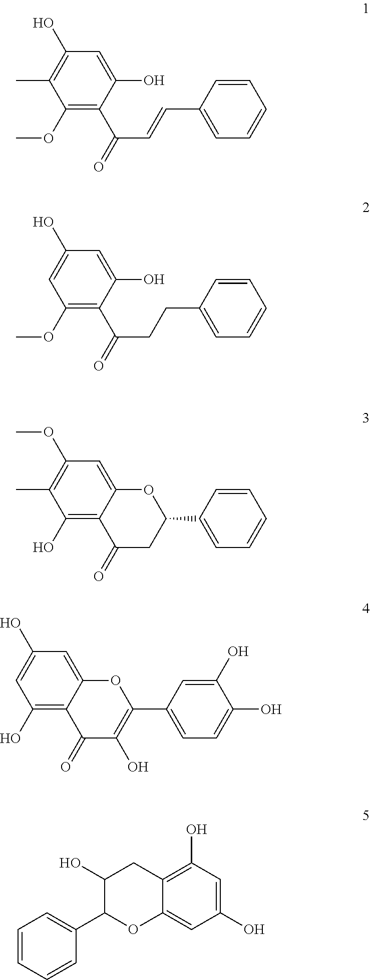 [SMILES: COc1c(C)c(O)cc(O)c1C(=O)/C=C/c1ccccc1 ; COc1cc(O)cc(O)c1C(=O)CCc1ccccc1 ; COc1cc2c(c(O)c1C)C(=O)C[C@@H](c1ccccc1)O2 ; O=c1c(O)c(-c2ccc(O)c(O)c2)oc2cc(O)cc(O)c12 ; Oc1cc(O)c2c(c1)OC(c1ccccc1)C(O)C2]

and derivatives thereof.

The extract may include or be enriched for any one or more of the compounds selected from Aurentiacin A, Cardamomin, 5-hydroxy-7-methoxy-6-methylflavanone, Quercetin, 3,5,7-trihydroxyflavan or derivatives of these compounds.

The extract may be prepared through a method which includes the steps of

collecting aerial plant material, comprising leaves and twigs of H. natalensis;

air drying the plant material;

grinding the plant material into a powder;

extracting by mixing the powder with alcohol;

filtering the extraction at least once; and

evaporating the solvent to retain the extract.

The method may include the further step of drying the extract, to produce a dried alcohol extract.

The oral care composition may include one or more essential oils. The one or more essential oils may include any one or both of Melaleuca alternifolia and Mentha piperita.

The oral care composition may include another plant extract. In one embodiment the plant extract may be in the form of green tea extract. The green tea extract may be in the form of concentrated green tea extract.

In one embodiment the oral care composition may include Melaleuca alternifolia essential oil, Mentha piperita essential oil, and concentrated green tea extract.

In an embodiment in which the microorganism is Prevotella intermedia, the concentration of the Heteropyxis natalensis extract may be at least 3.13 mg/ml, the concentration of the concentrated green tea extract may be at least 2 mg/ml, the concentration of Mentha piperita essential oil may be at least 0.05% (v/v), and the concentration of Melaleuca alternifolia essential oil may be at least 0.05% (v/v).

In an embodiment in which the microorganism is Candida albicans the concentration of the Heteropyxis natalensis extract may be at least 3.13 mg/ml, the concentration of the concentrated green tea extract may be at least 4 mg/ml, the concentration of Mentha piperita essential oil may be at least 0.05% (v/v), and the concentration of Melaleuca alternifolia essential oil may be at least 0.01% (v/v).

In an embodiment in which the microorganism is Streptococcus mutans the concentration of the Heteropyxis natalensis extract may be at least 0.78 mg/ml, the concentration of the concentrated green tea extract may be at least 0.78 mg/ml, the concentration of Mentha piperita essential oil may be at least 0.002% (v/v), and the concentration of Melaleuca alternifolia essential oil may be at least 0.0004% (v/v).

In an embodiment in which the in which the pathogenic oral microorganisms includes Streptococcus mutans, Prevotella intermedia and Candida albicans, the concentration of the Heteropyxis natalensis extract may be at least 3.13 mg/ml, the concentration of the concentrated green tea extract may be at least 4 mg/ml, the concentration of Mentha piperita essential oil may be at least 0.05% (v/v), and the concentration of Melaleuca alternifolia essential oil may be at least 0.05% (v/v).

The oral care composition may be formulated in an oral delivery system including any one of the group selected from capsules, tablets, mouth wash, gel, paste, toothpaste, impregnated dental floss, chewing gum or the like.

The disclosure also relates to the use of a Heteropyxis natalensis extract in the manufacturing of an oral care composition which inhibits the growth of potentially pathogenic oral microorganisms. The pathogenic oral microorganisms may include any one or more microorganisms selected from the group of Actinomyces israelli, Streptococcus mutans, Prevotella intermedia and Candida albicans.

The disclosure further relates to use of a composition which includes Heteropyxis natalensis extract, Melaleuca alternifolia essential oils, Mentha piperita essential oils and concentrated green tea extract in the manufacturing of an oral care composition for the treatment of periodontal disease by the composition's antioxidant activity.

There is further provided for a substance or composition comprising the oral care composition as described, for use in a method of treating periodontal disease.

The disclosure also extends to a further oral care composition for inhibiting the growth of potentially pathogenic oral microorganisms, said composition including Aurentiacin A or derivatives thereof.

The disclosure and disclosed embodiments also provide for use of Aurentiacin A or derivatives thereof for inhibiting the growth of potentially pathogenic oral microorganisms.

The disclosure and the disclosed embodiments further provide for a substance or composition comprising Aurentiacin A or derivatives thereof, for use in a method of treating periodontal disease.

The disclosure and disclosed embodiments also extends to a method of evaluating the attachment of microorganisms to an enamel surface of a tooth and the effect of a composition on such attachment, which includes

adding the composition to prepared enamel blocks;

adding microorganisms to the prepared enamel blocks; and

determining colonization of the microorganisms on the enamel through scanning electron microscopy (SEM).

In one embodiment the step of adding the composition to the prepared enamel blocks and the step of adding the microorganisms to the prepared enamel block may occur simultaneously.

In another embodiment the step of adding the microorganisms to the prepared enamel block occurs before the step of adding the composition to the prepared enamel blocks.

The step of adding the composition to the prepared enamel blocks and adding the microorganisms may occur after allowing a period of time to lapse.

The prepared enamel blocks may be obtained by extracting teeth from humans, sterilizing the teeth, removing the crowns of the teeth and cutting the crowns into blocks.

The various disclosed embodiments will now be described, by way of example only with reference to the following drawing(s) and table(s):

### BRIEF DESCRIPTION OF THE DRAWINGS AND TABLES

In the figure(s):

FIG. 1a shows an image of an enamel surface of the extracted tooth which was exposed to stimulated saliva to form a pellicle on the enamel;

FIG. 1b shows an enamel surface which was exposed to the plant extract, in accordance with an embodiment, in a carbohydrate containing growth medium (CASO), to determine if a coating similar to a normal pellicle, could be formed on the enamel (Treatment 1). This image shows the coating of the enamel surface of teeth by the plant extract;

FIG. 1c shows an image of an enamel surface coated with the chemical substance, Repelcote® that prevents adhesion of bacterial cells to a surface. The enamel was exposed to a carbohydrate medium to determine if a ‘pellicle’ could be formed on enamel;

FIG. 2 shows percentage inhibition of DPPH by Vitamin C;

FIG. 3 shows percentage inhibition of DPPH by Quercetin;

FIG. 4 shows percentage inhibition of DPPH by Heteropyxis natalensis;

FIG. 5 shows percentage inhibition of DPPH by concentrated green tea extract (TEAVIGO™);

FIG. 6 shows Percentage inhibition of DPPH by the synergistic compilation of H. natalensis (3.125 mg/ml), M. alternifolia (0.05% v/v), M. piperita (0.05% v/v) and concentrated green tea extract (TEAVIGO™) (2.5 mg/ml). These respective concentrations were taken as 100% initial concentration.

### DETAILED DESCRIPTION

The disclosed embodiments will be more readily understood through the following detailed description and scientific study, which is not intended to limit the invention, but merely exemplify the embodiments disclosed herein.

### Material and Methods

### Plant Material

Aerial plant parts, comprising of leaves and twigs of H. natalensis was collected. The plant was collected from the University of Pretoria's Botanical Garden during January. A voucher specimen was prepared and identified at the H.G.W.J. Schwelcherdt Herbarium (PRU), University of Pretoria, (PRU 096405).

### Preparation of Extract

The plant material was air dried at room temperature (25° C.), and ground to a fine powder using a Janke & Kunkel (IKA Labortechnik, Germany) grinder. The powdered material was extracted by shaking with 400 ml ethanol (Merck Chemicals (Pty) Ltd Wadeville, South Africa) (Labcom shaker). The sample was filtered through a Whattman No. 1 (110 mm diameter) (Merck Chemicals (Pty) Ltd Wadeville, South Africa) filter paper using a vacuum filter (Merck Chemicals (Pty) Ltd Wadeville, South Africa). The process was repeated several times. The solvent was evaporated in a BÜCHI Rotavapor (Labotec (PTY) Ltd. Halfway House, South Africa) under reduced pressure of 40° C. The extract was further dried at room temperature after which they were subjected to antimicrobial tests.

### Antimicrobial Activity

### Microbial Strains

The microorganisms used in this study included Actinomyces israelii (ATCC 10049), Prevotella intermedia (ATCC 25611), Streptococcus mutans (ATCC 25175), Candia albicans (ATCC 10231) and a strain of Candida albicans resistant to polyenes and azoles (1051604). The bacteria were grown on Casein-peptone Soymeal-peptone Agar medium (CASO) (Merck Chemicals (Pty) Ltd Wadeville, South Africa) under anaerobic conditions in an anaerobic jar with Anaerocult® A (Merck KGaA Darmstadt, Germany), at 37° C. for 72 hours. Actinomyces israelii and S. mutans had CASO agar enriched with 1% sucrose (Merck Chemicals (Pty) Ltd Wadeville, South Africa). The drug susceptible and drug resistant strains of C. albicans were grown on Sabouraud Dextrose 4% Agar (SDA) (Merck Chemicals (Pty) Ltd Wadeville, South Africa), at 37° C. for 72 hours. Sub-culturing was done every second week. Inocula were prepared by suspending bacterial test organisms in their respective broths until turbidity was compatible with McFarland Standard 1 (Merck Chemicals (Pty) Ltd Wadeville, South Africa). Yeast test organisms were suspended in sterile distilled water until turbidity was compatible with McFarland Standard 1 (McFarland, 1907).

### Determination of Minimum Inhibitory Concentration (MIC) and Minimum Bactericidal Concentration (MBC)

The microdilution technique using 96-well micro-plates, as described by Eloff (1998) was used to obtain the MIC and MBC values of the crude extracts against the microorganisms under study. The extracts, dissolved in 10% dimethyl sulphoxide (DMSO) (Merck Chemicals (Pty) Ltd), were serially diluted in broth (enriched for A. israelii and S. mutans) for the bacteria and sterile water for the Candida species; in the 96-well plate adding 48 hour old microorganisms grown at 37° C. The final concentration of extracts ranged from 12.5-0.10 mg/ml and the positive control, 5% chlorhexidine gluconate (CHX) (Dental Warehouse, Sandton, South Africa), ranged from 12.5-3.8×10⁻⁴ mg/ml. Amphotericin B (Davis Diagnostics, Gauteng) an established antifungal drug, ranging from 0.2 mg/ml to 1.5×10⁻³ mg/ml, was included for the Candida assays. The highest concentration of the solvent DMSO (2.5%) was found to be non-toxic to the microorganisms tested. Actinomyces israelii and S. mutans were incubated at 37° C., under anaerobic conditions, for 24 hours; P. intermedia was incubated at 37° C., under anaerobic conditions, for 48 hours; and C. albicans was incubated at 37° C. with p-iodonitrotetrazolium violet (INT) (Sigma-Aldrich, South Africa) already added, under moist aerobic conditions, for 24 hours.

To indicate bacterial growth, 50 μl of (0.2 mg/ml) INT, was added to micro-plate wells and incubated at 37° C., under anaerobic conditions, for 20-60 minutes until a red colour developed. The MIC was defined as the lowest concentration that inhibited the colour change of INT. The MBC was determined by adding 50 μl of the suspensions from the wells, which did not show any growth after incubation during MIC assays, to 150 μl of fresh broth. These suspensions were reincubated at 37° C. for 28 hours (48 hours for P. intermedia), under anaerobic conditions. The MBC was determined as the lowest concentration of extract which inhibited 100% growth of microorganisms (Cohen et al., 1998).

### Determination of Cytotoxicity

Microtitre plates with Vero and HEp-2 cells (Highveld Biological, Gauteng) were used for testing the four best ethanol extracts for cytotoxicity following the method of Basson (2005). Cytotoxicity was measured by the XTT (Sodium 3′-[1-(phenyl amino-carbonyl)-3,4-tetrazolim]-bis-[4 methoxy-6-nitro]benzene sulfonic acid hydrate) method using the cell proliferation kit II (Roche Diagnostics GmbH). A hundred microliters of Vero and HEp-2 cells (1×10⁵ ml) were seeded onto micro-plates and incubated for 24 hrs to allow the cells to attach to the bottom of the plate. Dilution series were made of the extracts and the various concentrations (400-3.1 μg/ml) were added to the micro-plate and incubated for 72 hours. The XTT reagents were added to a final concentration of 0.3 mg/ml and the cells were incubated for one to two hours. The positive drug controls Doxorubicin HCl (Sigma-Aldrich, South Africa) and Actinomycin D (Sigma-Aldrich, South Africa), at concentration ranges of 0.78-0.01 μg/ml, were included in the assay. After incubation the absorbance of the colour was spectrophotometrically quantified using an enzyme-linked immunosorbent assay (ELISA) plate reader (BIO-TEK Power-Wave XS, Weltevreden Park, South Africa), which measured the optical density at 490 nm with a reference wavelength of 690 nm. The assay was carried out in triplicate.

### Synergistic Assay

Aerial plant parts, comprising of leaves and twigs of H. natalensis was collected. The plant was collected from the University of Pretoria's experimental farm during January. A voucher specimen was prepared and identified at the H.G.W.J. Schwelcherdt Herbarium (PRU), University of Pretoria, (PRU 096405). Melaleuca alternifolia essential oil (Holistic Emporium cc, Gauteng, South Africa), Mentha piperita essential oil (Holistic Emporium cc, Gauteng, South Africa), and TEAVIGO™ (Chempure (Pty) Ltd, Silverton, South Africa), were purchased as well for the present investigation.

The H. natalensis plant material was air dried at room temperature (25° C.), and ground to a fine powder using a standard food processor. The powdered material was extracted with ethanol (Merck Chemicals (Pty) Ltd Wadeville, South Africa) under pressure (100 bar) and regulated temperature of 50° C. in a BUCHI Speed Extractor, E-916 (BUCHI Labortechnik AG, Switzerland). The solvent was evaporated on low boiling point in a Genevac, EZ-2 plus (Genevac SP Scientific, UK), after which the extract was subjected to antimicrobial tests.

The microorganisms used in this study included Prevotella intermedia (ATCC 25611), Streptococcus mutans (ATCC 25175) and Candida albicans (ATCC 10231). The bacteria were grown on Casein-peptone Soymeal-peptone Agar) (CASO) (Merck Chemicals (Pty) Ltd Wadeville, South Africa) enriched with 1% sucrose (Merck Chemicals (Pty) Ltd Wadeville, South Africa) under anaerobic conditions in an anaerobic jar with Anaerocult® A (Merck Chemicals (Pty) Ltd Wadeville, South Africa), at 37° C. for 48 hours. Candida albicans was grown on Sabouraud Dextrose 4% Agar (SDA) (Merck Chemicals (Pty) Ltd Wadeville, South Africa), at 37° C. for 48 hours. Sub-culturing was done every second week. Inocula were prepared by suspending bacterial test organisms in their respective broths until turbidity was compatible with McFarland Standard 1 (Merck Chemicals (Pty) Ltd Wadeville, South Africa) (McFarland, 1907).

To determine the effects of combinations of H. natalensis, M. alternifolia essential oil, M. piperita essential oil and TEAVIGO™, the MIC of each component was determined first using the anti-microbial microplate method of Eloff (1998). A stock solution of the ethanol extract of H. natalensis was prepared in 20% dimethyl sulphoxide (DMSO) (Merck Chemicals (Pty) Ltd); while TEAVIGO™ was dissolved in distilled water. The stock solutions were serially diluted in enriched Casein-peptone Soymeal-peptone medium broth (Merck Chemicals (Pty) Ltd) for the bacteria and Sabouraud Dextrose 4% broth (Merck Chemicals (Pty) Ltd) for Candida; in the 96-well plate adding a McFarland Standard 1 inoculum of 48 hour old microorganisms grown at 37° C. The final concentration of the extract and TEAVIGO™ ranged from 0.10-12.5 mg/ml and the positive control, 1.25% v/v chlorhexidine gluconate (CHX) (Dental Warehouse, Sandton, South Africa), ranged from 4.77×10⁻⁶-0.31% v/v. The essential oils were dissolved in 10% Tween (80) (Merck Chemicals (Pty) Ltd Wadeville, South Africa). The final concentration tested of the essential oils ranged from 1.6×10⁻⁵-1.25% v/v. The highest concentration of the solvent Dimethyl sulphoxide (DMSO) (5%) and Tween 80 (2%) was found to be non-toxic to the microorganisms tested. The inoculated plates were incubated at 37° C., under anaerobic and aerobic conditions respectively for 24 hours before adding the colour indicator PrestoBlue (Lail et al., 2013). The minimum inhibitory concentration (MIC) was defined as the lowest concentration that inhibited the colour change of PrestoBlue.

The synergistic activity of the samples was determined using a modified checkerboard method. This process was repeated for all combinations of the 4 agents for each microorganism tested.

### Anti-Adherence

### Cytokine Assay

Levels of IL-8 in the supernatants were determined using an enzyme-linked immunosorbent assay (ELISA) kit (Pharmigen, OptEIA Human IL-8 Set, catalog no. 555244) obtained from BD Bioscience. Cells were grown in pre-coated T-75 flasks in Eagle's minimum essential medium (MEM) containing 10% fetal bovine serum (FBS) and re-fed every 2-3 days until confluent. The confluent, adherent monolayers were then released from the plastic surface after treatment with polyvinyl-pirrolidone (PVP)-trypsin-EDTA and were seeded to 24-well plates for 24 hrs before receiving the treatments. The cells were rinsed three times with phosphate-buffered saline (PBS) buffer and 1 ml antibiotic free medium was added to the cells which were then treated with H. natalensis at non-cytotoxic concentrations established previously in cell culture studies.

To determine if the plant extract had an effect on IL-8 release from oral epithelial cells, extracts were added to the cells at concentrations varying from 12.5-200 μg/ml. Time-dependent studies were carried out. Plant extracts were added to HEp-2 cells and one hour later A. israelii was added. Plant extracts and A. israelii were added to HEp-2 cells together and lastly A. israelii was added to HEp-2 cells with the plant extracts being added one hour later. Heteropyxis natalensis extracts were added in duplicate to each well. A negative control of only A. israelii and HEp-2 cells was included. The plates were incubated overnight at 37° C. in 5% CO₂.

After the treatment described above, the supernatants were collected to determine IL-8 released using a commercially available ELISA kit. Briefly, 96-well plates were coated with 100 μl of capture antibody (anti-human IL-8 monoclonal antibody) incubated overnight, washed three times with 0.05% Tween-20 in FBS and coated with PBS with 10% FBS in order to block non-specific binding. Known concentrations of IL-8 (standard) and the samples containing the IL-8 released by the cells after treatment (supernatant) were added as aliquots into appropriate wells, incubated for two hours and decanted from the wells. Aspirated and washed five times. Biotinylated (the attachment of a biotin residue to a biological macromolecule in order to label it) anti-human IL-8 monoclonal antibody and streptavidin-horseradish peroxidase conjugate were added and incubated for one hour. After washing the plate, a solution containing a substrate for the enzyme (3,3′,5,5′ tetramethylbenzidine-peroxide chromogen) present in the anti-IL-8 and enzyme reagent mixture was added and the plate was incubated for 30 minutes. The reaction was stopped using a 2N sulfuric acid (H2504) solution and the absorbance was read at 450 nm using an ELISA plate reader (BIO-TEK Power-Wave XS, Weltevreden Park, South Africa). The absorbencies were then used to calculate the IL-8 concentration from the standard curve (Vilela et al., 2006).

### Ultrastructure

To determine whether or not the plant extract affected the attachment of S. mutans to the enamel of human teeth, time-dependent studies were carried out. Firstly H. natalensis extract, at an MIC concentration of 1.82 mg/ml and sub-MIC value of 0.91 mg/ml, were added to the prepared enamel fragments and one hour later S. mutans was added. Secondly plant extracts and S. mutans were added to the enamel fragments together and lastly S. mutans was added to the enamel fragments with the plant extracts being added one hour later. The plant extract was added in duplicate to each well. The positive control consisted of enamel fragments being coated with a chemical that prevents adhesion of bacteria. A negative control of only S. mutans and enamel fragments was included. The plates were incubated overnight.

Teeth collected for this study had been extracted from human patients for reasons other than the purpose of this study. Each patient who attended the extraction clinic of the University of Pretoria must complete and sign a patient information leaflet and informed consent form. Non-carious, recently extracted human teeth, were collected from Dental Clinics. Only teeth which had been extracted for periodontal or orthodontic reasons were used. Ethical and safety guidelines for the handling of human teeth and laboratory research were strictly followed. Immediately after extraction the teeth were rinsed in running water. Thereafter the teeth were placed in distilled water in an ultrasonic water bath and sonicated for periods of fifteen minutes in clean distilled water until all loose biological material was removed and stored at 4° C. The crowns of the teeth were removed by horizontally sectioning at the cemental-enamel junction with a diamond wafering blade in an Isomet 11-1180 low speed saw (Buehler Ltd., Lake Bluff, Ill., USA) under permanent water irrigation. The crowns were further cut into blocks; the samples were placed in sterile Ringer's solution (Merck SA (Pty) Ltd., Halfway House, South Africa) and sterilized at 125° C. for 15 min.

The rest of the procedure was carried out under sterile conditions. Sterility was maintained for the duration of the entire experiment that was conducted in a positive sterile airflow laboratory, using sterile instruments as well as gloves and masks. Before sterilisation some of the enamel blocks used as samples were cleaned, dried and coated with 2% Dimethyl dichlorosilane in 1,1,1-trichloroethane (Repelcote®—Saarchem-Holpro Analytic (Pty.) Ltd., 40 Fransen Street, Chamdor, Krugersdorp) in order to create a repellent surface that would prevent organisms attachment, utlized as a positive control.

All samples were placed in 24 well tissue culture plates containing 1 ml CASO broth and incubated at 37° C. for 1 hour. For Treatment 1, 1 ml of the different plant extracts were added and incubated at 37° C. for 1 hour, after which 1% MacFarland Standard-1 bacterial suspension was added. For Treatment 3, the bacterial suspension was added to the enamel blocks and incubated for an hour, after which the different plant extracts were added. For Treatment 2, the different plant extracts and the 1% MacFarland Standard-1 bacterial suspension were added at the same time, the plates incubated anaerobically as described in a shake incubator for 24 and 48 hours. One enamel block with the organism tested was used as a positive control and all the samples were prepared for the Scanning Electron Microscope (SEM) as the positive control.

### Preparation for Scanning Electronmicroscopy (SEM)

One sample was collected from each of the tissue culture wells containing the different plant extract concentrations at 24 and 48 hrs for SEM to determine the colonization of the organisms on enamel. The samples were prepared according to standard methods for biological SEM evaluation according to Glauert (1975) and Hayat (1981).

### Purification of Active Compounds

The dried ethanolic extract (60 g) was subjected to fractionation on a silica column (10×70 cm) using a gradient of hexane:ethyl acetate of increasing polarity (0% to 100% ethyl acetate) as eluent. Twenty-nine fractions were collected and those with similar thin layer chromatography (TLC) profiles were combined together (TLC plates were developed using hexane:ethyl acetate (7:3); hexane:ethyl acetate (8:2); dichloromethane:methane (99.5:0.5) and dichloromethane:methane (99:1) as eluent. Acidic vanillin; 0.34% vanillin in 3.5% sulphuric acid in methanol; was used for detection). Thirteen major fractions (1B to 13B) were obtained and tested for antibacterial activity against A. lsraelii (Table 1).

TABLE 1
Average minimum Inhibitory concentration (MIC) and minimum
bactericidal concentration (MBC) of the Fractions

| Fraction | MIC (mg/ml) | MBC (mg/ml) |
|---|---|---|
| 1B | >12.5 | Na^{a} |
| 2B | >12.5 | Na |
| 3B | >12.5 | Na |
| 4B | >12.5 | Na |
| 5B | 9.375 | 9.375 |
| 6B | 9.375 | 9.375 |
| 7B | 0.830078 | 1.611328 |
| 8B | 0.830078 | 2.34375 |
| 9B | 1.5625 | 2.34375 |
| 10B | 2.213542 | 8.203125 |
| 11B | 4.25 | 12.5 |
| 12B | 12.5 | Na |
| 13B | 1.822917 | 5.46875 |
| Positive control^{b} | 0.018311 | 0.048828 |

^{a}Na: Not active;
^{b}Chlorhexidine

Based on the antibacterial results and preliminary TLC plates; Fractions 7B (2 g), 9B (3 g), 11B (0.8 mg) and 12B (1 g) were chromatographed separately using Sephadex columns (Sigma-Aldrich, South Africa). Fraction 7B was chromatographed using dichloromethane:methane (99.5:0.5). Sixty-four subfractions were collected, spotted on TLC plates and developed in dichloromethane:methane (99:1). Pure compound 1 was obtained. Fraction 9B was chromatographed using 100% ethanol. Ninety-one subfractions were collected, spotted on TLC plates and developed in dichloromethane:methane (99.5:0.5). Pure compounds 2 and 3 were obtained. Fraction 11B was chromatographed using dichloromethane:methane (99:1). Fifty-five subfractions were collected, spotted on TLC plates and developed in dichloromethane:methane (99:1). Pure compound 4 was obtained. Fraction 12B was chromatographed using dichloromethane:methane (99:1). Seventy-one subfractions were collected, spotted on TLC plates and developed in dichloromethane:methane (99:1). Pure compound 5 was obtained.

### Antioxidant Assay

Evidence suggests an association between periodontal diseases and an imbalance between oxidants and antioxidants due to both an increase in free radical production and a decrease in the antioxidant activity of saliva. Reactive oxygen species (ROS) have been linked to the destruction of periodontal tissues (Alviano et al., 2008).

DPPH (2,2-diphenyl-1-picrylhydrazyl hydrate) is a stable free radical with an unpaired valence electron at a nitrogen atom forming a bridge. The free radical can initiate a chain reaction which causes the removal of an electron from another molecule to complete its own orbital. This free valence electron forms the basis of the DPPH assay as samples are tested to determine the scavenging capability of this DPPH radical.

A modified antioxidant assay was used (du Toit et al., 2001). A 2% stock solution of M. piperita and M. alternifolia, a 2 mg/ml stock solution of ascorbic acid (vitamin C) (Sigma-Aldrich (Pty) Ltd, Aston Manor, South Africa) and H. natalensis, a 1 mg/ml stock solution of Quercetin, a 500 μg/ml stock solution of TEAVIGO™ and a synergistic compilation of H. natalensis (3.125 mg/ml), M. alternifolia (0.05% v/v), M. piperita (0.05% v/v) and TEAVIGO™ (2.5 mg/ml) were prepared.

In a 96 well ELISA plate, 20 μl of each sample was added to 200 μl dH₂O (except for Quercetin which was added to 200 μl EtOH), serial dilutions were done. After which 90 μg/ml DPPH (Sigma-Aldrich (Pty) Ltd, Aston Manor, South Africa) dissolved in ethanol (40 μg/ml) were added to all wells. All samples were prepared in triplicate. Vitamin C was used as a positive control, an extract control and blank controls were also done. The absorbance was measured in an ELISA plate reader at 515 nm (du Toit et al., 2001).

### Results and Discussion

The MIC exhibited by the ethanol extract of H. natalensis was found to be 1.82 mg/ml and 0.88 mg/ml against S. mutans and A. israelii respectively. The plant exhibits moderate toxicity against HEp-2 cells with a 50% inhibition (1050) of 33.66±0.04 μg/ml (Table 2)

TABLE 2
The minimum inhibitory concentration (MIC), minimum bactericidal concentration (MBC) and 50% inhibitory
concentration (IC₅₀) of the ethanol extracts of selected plants on oral microorganisms

| | Microorganism tested | | | | | | | | | | Cytotoxicity | |
|---|---|---|---|---|---|---|---|---|---|---|---|---|
| | MIC (mg/ml) | | | | | MBC (mg/ml) | | | | | IC₅₀ (μg/ml)— | |
| | Gram +ve | | Gram −ve | Yeast | | Gram +ve | | Gram −ve | Yeast | | Standard deviation | |
| Plant extract | A.i | S.m | P.i | C.a | C.a (res) | A.i | S.m | P.i | C.a | C.a (res) | Vero | HEp-2 |
| Heteropyxis | 0.88 | 1.82 | 3.13 | 10.94 | 12.5 | 3.32 | 3.13 | >12.5 | >12.5 | >12.5 | 147 ± | 33.66 ± |
| natalensis | 0.033^{b} | 0.061^{b} | 0.26^{b} | 0.352^{b} | 0.26^{b} | 0.039^{b} | 0.415^{b} | 1.042^{b} | 0.293^{b} | 0.293^{b} | 0.150 | 0.04 |
| Positive | | | | >0.2^{c} | 0.013^{c} | | | | | 0.013^{c} | 0.06 ± | 8.5 × 10⁻³ ± |
| controls | | | | | | | | | | | 2.44^{d} | 9.^{95×10-5(e)} |

^{a}Na: not active;
^{b}Chlorhexidine;
^{c}Amphotericin B;
^{d}Doxorubicin;
^{(e)}Actinomycin D;
A.i: Actinomyces israelii;
S.m: Streptococcus mutans;
P.i: Prevotella intermedia;
C.a: Candida albicans;
C.a (res): Polyene and azole resistant C. albicans

### Synergistic Assay

The modified checkerboard method was utilized for the reduction of MIC values. This method provided numerous concentration variables for the agents under investigation and their inhibition potential.

TABLE 3
Comparison of the minimum inhibitory concentrations of the tested agents alone
and in combination after utilizing statistical analyses

| | P. intermedia | | C. albicans | | S. mutans | |
|---|---|---|---|---|---|---|
| | Agent alone | Combination | Agent alone | Combination | Agent alone | Combination |
| H. natalensis (mg/ml) | 12.50 | 3.13 | 8.33 | 3.13 | 2.60 | 0.78 |
| TEAVIGO ™ (mg/ml) | >12.5 | 2.00 | 10.42 | 4.00 | 1.30 | 0.78 |
| M. piperita (% v/v) | 0.20 | 0.05 | 0.10 | 0.05 | 0.10 | 2 × 10⁻³ |
| M. altemifolia (%v/v) | 0.29 | 0.05 | 0.24 | 0.01 | 0.29 | 4 × 10⁻⁴ |

There is a reduction in the MIC values of each agent alone, when used in combination for each of the microorganisms tested (Table 3). And therefore we can state that there is an overall increase in the inhibitory activity when the agents are used in combination.

### Cytokine Assay

Although the plants themselves did not induce a release of IL-8 (not shown on graph) there seems to be a negative interaction between H. natalensis and A. israelii that induces the release of IL-8, as all the IL-8 readings are higher than that of the negative control of the bacteria and cells only, at 4.6 μg/ml. Although H. natalensis inhibited A. israelii growth at 0.88 mg/ml and is bacteriostatic at 3.32 (mg/ml); it does not appear to hinder the adhesion mechanism of A. israelii and must therefore affect the microorganism in another way.

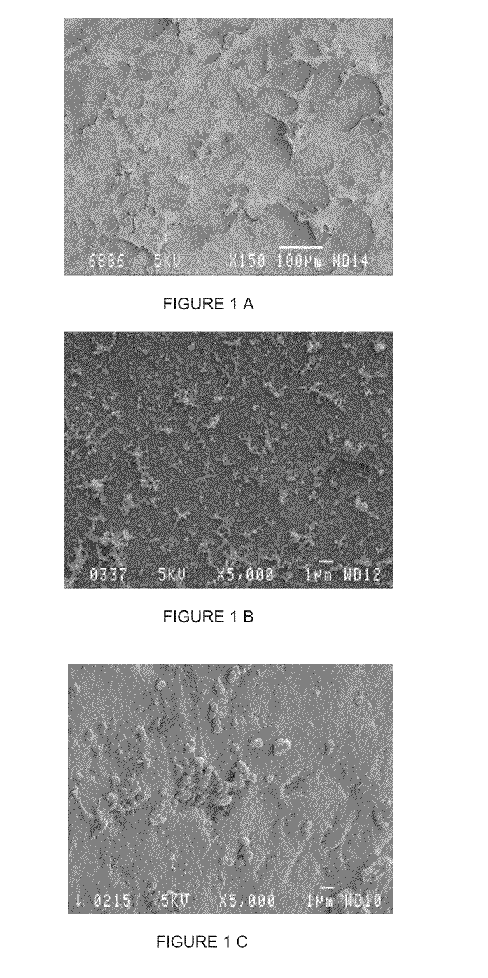

FIG. 1a, b and c represents the comparison of the pellicle as formed on the enamel surface of teeth.

### Identification of the Isolated Compounds

The identification of the compounds was done by ¹H and ¹³C nuclear magnetic resonance (NMR) and distortionless enhancement by polarization (DEPT). The MIC, MBC and IC₅₀ of the isolated compounds against Actinomyces israelii and on HEp-2 cells are shown in Table 4.

TABLE 4
The minimum inhibitory concentration (MIC), minimum bactericidal
concentrations (MBC) and 50% inhibition of cell growth (IC₅₀) of the isolated compounds
against Actinomyces israelii and on HEp-2 cells

| Isolated compounds | MIC(mg/ml) | MBC (mg/ml) | IC₅₀ (μg/ml) + S.D. |
|---|---|---|---|
| Heteropyxis natalensis | 1.5625 | 1.5625 | 33.66 ± 0.04 |
| 5-hydroxy-7-methoxy-methylflavanone (3) | — | — | — |
| Aurentiacin A (1) | 0.0625 | 0.0625 | 6.36 ± 5.095 |
| Cardamomin (2) | >1 | NA^{a} | 52.12 ± 9.41 |
| 3,5,7-trihydroxyflavan (5) | >1 | NA | >200 |
| Quercetin (4) | 1 | 1 | 186.7 ± 42.605 |
| Positive control | 0.024^{b} | 0.024^{b} | 9.6 × 10⁻³ ± 0.0003^{c} |

^{a}NA: Not active;
^{b}Chlorhexidine;
^{c}Actinomycin D

### Fraction 9B Subfraction 2-5, Sub-Subfraction 4

Compounds 2 and 3 were recognised as chalcones with different substitution patterns in ring B, while ring C is not substituted. Compound 3 was identified based on the spectral data of ¹H and ¹³C NMR. It showed in ¹H NMR signals of an unsubstituted ring C at 7.72 (2H 2, 6), 7.45 (3H, 3, 4, 5). It also showed signals of trans alkene at 8.05 and 7.73 (1H, d each, J=15.4 Hz) and two aromatic protons at 6.08, 6.02 (s each, H3′ and 5′) in addition to a methoxyl signal at 3.97.

The ¹³C NMR data showed 16 carbon signals including a carbonyl group at 193.0 ppm. DEPT-135 showed 10 protonated carbons one of them is the methoxyl group at δ_{C} 56.4 and two α,β double bonds adjacent to the carbonyl group at 142.6 and 128.4; while the other signals were attributed to the aromatic carbons of both ring B and ring C.

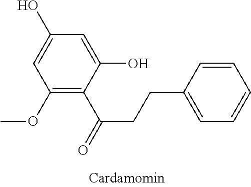 [SMILES: COc1cc(O)cc(O)c1C(=O)CCc1ccccc1]

The data given established the structure of Cardamomin, or cardamonin, as compound 2. Cardamomin is a 2′-Me ether derivative of 2′,4′,6′-Trihydroxychalcone and has been previously isolated from Alpinia katsumadai, Boesenbergia pandurata, Comptonia peregrina, Myrica pensylvanica, Piper sp., Populus sp. and Dracaena draco. Cardamomin has been shown to inhibit pro-inflammatory mediators and therefore has anti-inflammatory activity and has also shown antitumor activity (Dictionary of Natural Products, 2011).

### Fraction 7B Subfraction 26 Sub-Subfraction 30

¹H NMR showed downfield signals at 6H 13.51 ppm of a chelated hydroxyl at C-6′; signals of monosubstituted benzene ring at 7.40 (s, 3H), 7.67 (s, 2H) and another singlet signal of an aromatic proton at 6.31, two doublets at 8.02, 7.79 (d, J=15.8 Hz), a low field methyl group at 2.09 and a methoxyl group at 3.67.

The ¹³C NMR data showed 17 signals; two methyls (60.9,7.2), eight methines at 127.1 (C-2,6), 128.3 (C-3,5), 125.9 (C-4), 142.0 (C-7), 129.6 (C-8) and 98.6 (C-3) and a carbonyl group at 191.9 (C-9) in addition to seven quarternary carbons.

The above data indicated the presence of a chalcone derivative with no oxygenation at ring B. Ring A showed only one signal of an aromatic proton (OH 6.31) which showed correlation heteronuclear multiple bond correlation (HMBC) to C-1′ (107.3) and C-3′ (110.1). The hydroxyl proton (OH 13.51) showed correlation with C-1′ and C-5′ H-5′/C6′, C1′, C3′ and C4′. This relation could establish the substitution pattern of ring A to 2′-methoxy,4′,6′ dihydroxy. The compound (1) was identified as 2′-Me ether derivative of 2′,4′,6′-trihydroxy-3′-methylchalcone; or more commonly known as aurentiacin A.

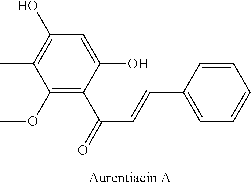 [SMILES: COc1c(C)c(O)cc(O)c1C(=O)/C=C/c1ccccc1]

Aurentiacin A has previously been isolated from Didymocarpus aurentiacum, Comptonia peregrina, Dalea species including Dalea scandens var. paucifolia (medicinal value in Mexico), Myrica pensylvanica and also from Dracaena species (Dictionary of Natural Products, 2011). Aurentiacin A, isolated from Myrica serrate, inhibits the growth of Cladosprium cucumerinum, Bacillus subtilis and E. coli (Dominguez et al., 1980; Gafner et al., 1996).

### Fraction 9B Subfraction 2-5, Sub-Subfraction 2

The compound showed to be C-flavonoid from its spectroscopic data. The ¹H NMR showed signals 5.44 (d, J=14.2 Hz) of H2. Two H-3 protons appeared at 2.64 (d, J=16.6 Hz) and 3.04 (dd, 16.6, 14.2 Hz). The signal at 6.35 (s) was attributed to H-8 and those at 7.40 and 7.53 attributed to monosubstituted ring B, methyl (attached at C-6) appears at 2.04 and a methoxyl group at 3.76. ¹³C NMR and DEPT-135 showed a two methyl signals at 61.0 and 8.0, a methylene group at 45.9 and 5 methine signals at 79.3 (C2), 100.0 (C8), 126.6, 129.1 and 129.2 of ring B. Other signals for quaternary carbons belong to C-4 (188.0), C-7 (163.4), C-5 (162.9), C-9 (116.5), C1′ (140.4) and C-6 (113.7).

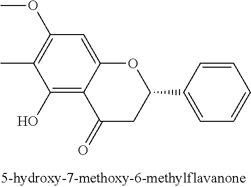 [SMILES: COc1cc2c(c(O)c1C)C(=O)C[C@@H](c1ccccc1)O2]

The above mentioned data established the structure of the compound (3) as 5-hyroxy-7-methoxy-6-methylflavanone. Previously isolated from Leptospermum scoparium (used in Australian and New Zealand traditional medicine), Leptospermum recurvum, Piper carniconnectivum, Pseudotsuga wilsoniana (used as building wood in Taiwan), the seeds of Myrica gale (fruit is a beer additive, essential oil is an insect repellent) and a trace constitute of Pityrogramma triangularis (Dictionary of Natural Products, 2011; Facundo & Braz-Filho, 2004; Fang et al., 2011; Hsieh et al., 1997; Mayer, 1989; Mustafa et al., 2003).

### Fraction 12B Subfraction 56, Sub-Subfraction 70

The ¹H NMR showed five aromatic signals at 6.80 (m), two meta coupled protons at 5.92, 5.84 (br. s each), two protons germinal to hydroxyl groups at 4.55 (d, J=7.0 Hz), 3.36 (m) [C-2, C-3], in addition to methylene protons at 2.84 (dd, J=16.1, 4.8 Hz) and 2.49 (dd, J=16.1, 8.4 Hz).

This compound has two possibilities, depending on the alpha D value.

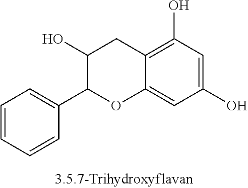 [SMILES: Oc1cc(O)c2c(c1)OC(c1ccccc1)C(O)C2]

The above ¹H NMR data established the structure of compound 5 as 3,5,7-Trihydroxyflavan. There are two variants of the compound, namely the 2R, 3R and 2R, 3S forms. The 2R, 3R form is known as distenin, previously isolated from Dennstaedtia distenta; while the 2R, 3S form is known as 3-Oxykoaburagenin, previously isolated from the leaves of Enkianthus nudipes (Dictionary of Natural Products, 2011).

### Fraction 11 Subfraction 10

This compound was identified as the well-known flavonoid, quercetin. The ¹H NMR showed a typical quercetin signal at 7.82 (br. s, H-2′), 7.70 (br. d, J=8.0 Hz, H-6′), 6.99 (br. d, J=8.0 Hz, H-5′), 6.52 (br. s, H-8) and 6.26 (br. s, H-6).

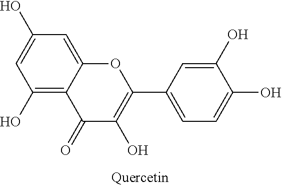 [SMILES: O=c1c(O)c(-c2ccc(O)c(O)c2)oc2cc(O)cc(O)c12]

The above mentioned data established the structure of the compound (4) as 3,3′,4′,5,7-pentahydroxyflavone, or more commonly known as quercetin. This compound has been previously isolated from many plant species, especially fruits, such as Helichrysum, Euphorbia and Karwinskia spp. The compound is present in almost all species of the Umbelliferae family and is present in the Solanaceae, Rhamnaceae and Passifloraceae families (Dictionary of Natural Products, 2011; Geoghegan et al., 2010; Muanda et al., 2011).

### Antioxidant Assay

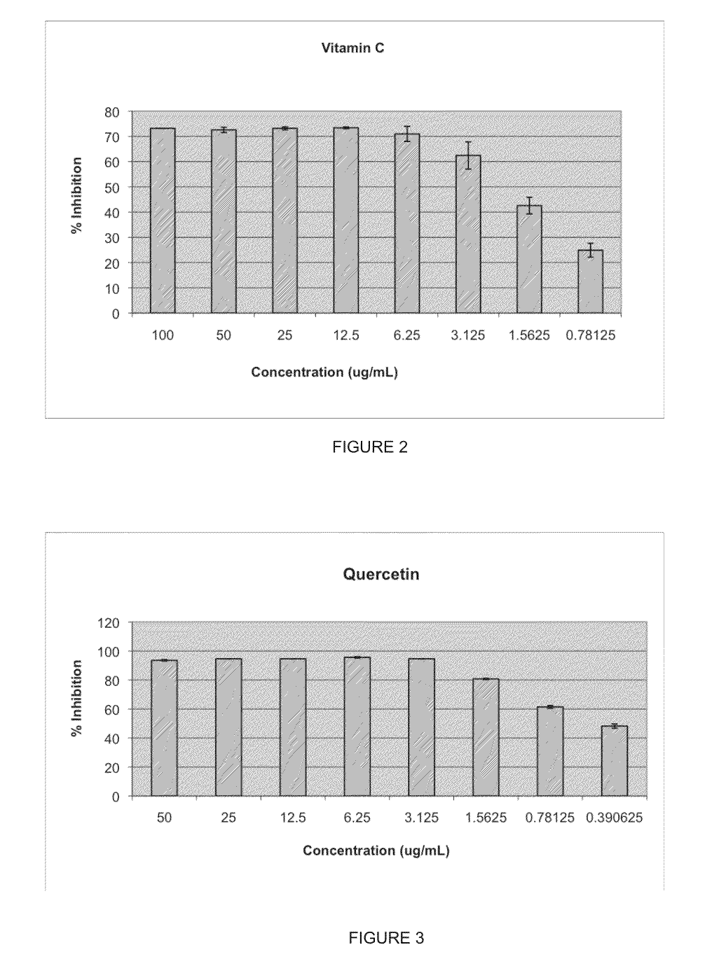

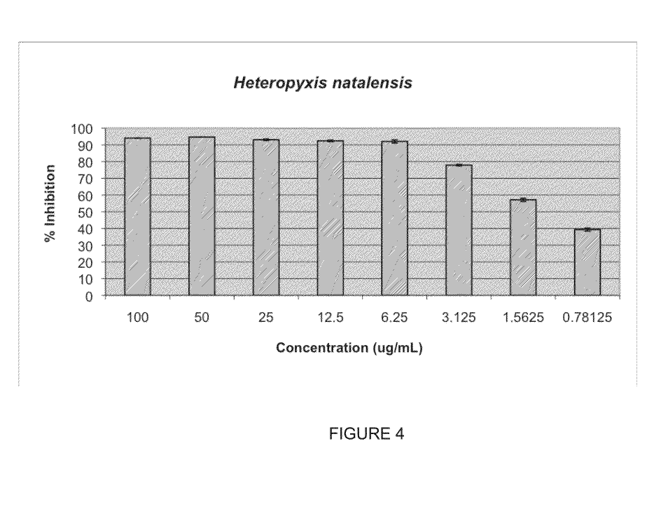

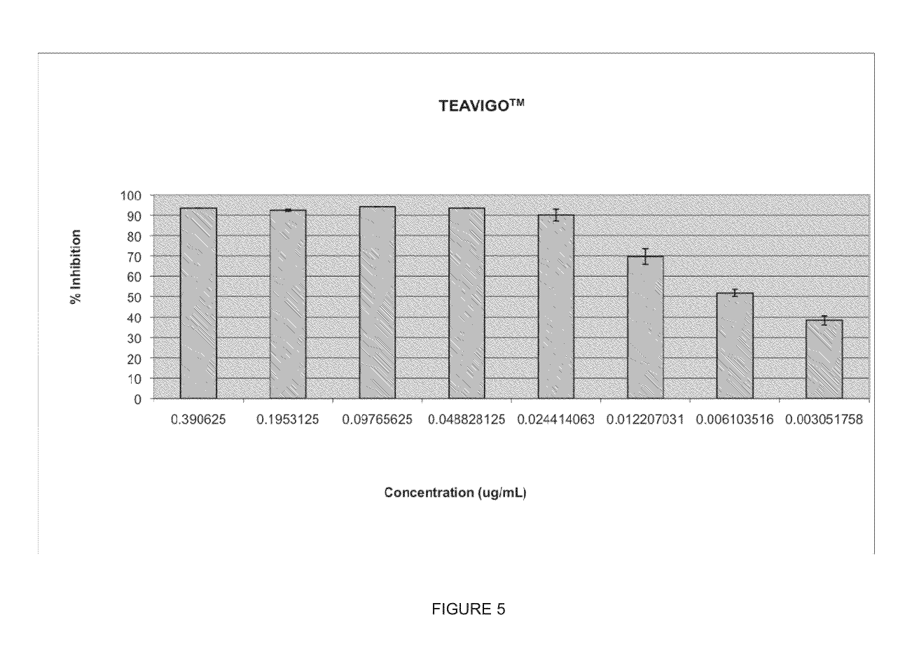

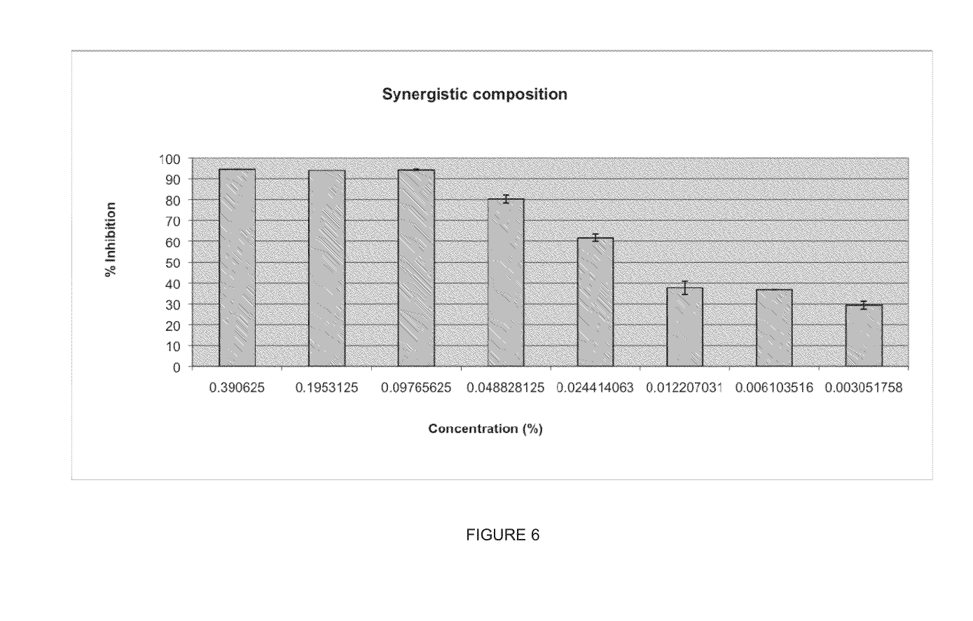

The percentage inhibition of DPPH, where DPPH turned colourless, was graphically determined as shown in FIGS. 2 to 6.

In a study conducted by Muchuweti et al. (2006), a 70% ethanol extract of the leaves and twigs of H. natalensis at a concentration of 32.26 μg/ml had a 29.65 inhibition percentage. A much higher inhibition percentage (>90%) is obtained in this investigation at the same concentration.

Melaleuca alternifolia and M. piperita did not exhibit inhibition of DPPH at the highest concentration tested (0.1% v/v). The IC₅₀ is 50% of DPPH that is inhibited/turned colourless by the samples tested and was calculated using GraphPad Prism 4 (San Diego, Calif., USA).

TABLE 5
50% of DPPH that is inhibited by the samples tested

| Component | IC₅₀ (μg/ml) ± SD | Estimated individual IC₅₀: | |
|---|---|---|---|
| Vitamin C | 1.98 ± 0.006 | | |
| Quercetin | 0.415 ± 0.01095 | | |
| Mentha piperita | >0.10% v/v | | |
| Melaleuca alternifolia | | | |
| Heteropyxis natalensis | 1.015 ± 0.0122 | | |
| TEAVIGO ™ | 0.005329 ± 0.00026 | | |
| Synergistic composition: | 0.01774 ± 0.00056 | | |
| H. natalensis | | 0.381 < × > 0.763 | μg/ml |
| TEAVIGO ™ | | 0.305 < × > 0.610 | μg/ml |
| M. piperita | | 6.1 × 10⁶ < × > 1.22 × 10⁵ % | v/v |
| M. alternifolia | | 6.1 × 10⁶ < × > 1.22 × 10⁵ % | v/v |

Heteropyxis natalensis, Quercetin and TEAVIGO ™ all exhibit lower IC₅₀ values than the positive control vitamin C (1.98 ± 0.006 μg/ml). According to du Toit et al. (2001), greater antioxidant and free radical savaging activities are exhibited by flavonoids than vitamins C and E on an equimolar basis. As Quercetin is a flavonoid and H. natalensis has been shown to contain several other flavonoids, this may explain why these two samples show better antioxidant activity than vitamin C. The IC₅₀ value of the synergistic composition is given as 100% representing the highest values tested in combination, namely H. natalensis at 3.125 mg/ml, M. alternifolia and M. piperita at 0.05% and TEAVIGO ™ at 2.5 mg/ml. The estimated IC₅₀ values are also given for each individual component of the synergistic combination.

### CONCLUSION

Heteropyxis natalensis exhibited activity against the Gram-positive microorganisms, A. israelii and S. mutans and the Gram-negative bacteria, P. intermedia. Heteropyxis natalensis exhibited moderate cytotoxicity. The cytokine, IL-8, levels were not reduced when the extract of H. natalensis was utilized to prevent the interaction of A. israelii with the epithelial cells, HEp-2. Heteropyxis natalensis interferes with pellicle formation and glucan binding of S. mutans to the enamel surface of the tooth. Five previously isolated compounds were identified for the first time from the ethanolic extract of H. natalensis leaves and twigs. The compounds were identified as Aurentiacin A (1), Cardamomin (2), 5-hydroxy-7-methoxy-6-methylflavanone (3), Quercetin (4) and 3,5,7-trihydroxyflavan (5). The MICs of the compounds 1 and 4 were found to be 0.0625 mg/ml and 1 mg/ml respectively against A. israelii. Compounds 2 and 5 exhibited no activity under 1 mg/ml against A. israelii. From the antioxidant assay it was shown that Heteropyxis natalensis has exhibited remarkable antioxidant activity on its own and appears to have even better activity in the synergistic composition. This plant extract may therefore aid in preventing periodontal diseases due to an imbalance between oxidants and antioxidants. The synergistic assay showed that there is an overall increase in inhibitory activity when H. natalensis extract is used in combination with Melaleuca alternifolia essential oil, Mentha piperita essential oil, and concentrated green tea extract.

1. An oral care composition for inhibiting the growth of potentially pathogenic oral microorganisms, said composition comprising
an extract of Heteropyxis natalens prepared by drying aerial plant material of H. natalensis, grinding the plant material into a powder, extracting by mixing the powder with a solvent to produce an extraction, filtering the extraction and evaporating the solvent to retain the extract;
the composition being formulated in an oral delivery system including any one of the group selected from capsules, tablets, mouth wash, gel, paste, toothpaste, impregnated dental floss and chewing gum.

2. The oral care composition as claimed in claim 1, in which the extract includes or is enriched for any one or more of the compounds selected from Aurentiacin A, Cardamomin, 5-hydroxy-7-methoxy-6-methylflavanone, Quercetin, 3,5,7-trihydroxyflavan or derivatives of these compounds.

3. The oral care composition as claimed in claim 1, in which the composition includes the extract of Heteropyxis natalensis and one or more essential oils.

4. The oral care composition as claimed in claim 3, in which the one or more essential oils includes any one or more selected from the group of Melaleuca alternifolia and Mentha piperita.

5. The oral care composition as claimed in claim 1, in which the composition includes one or more other plant extracts.

6. The oral care composition as claimed in claim 5, in which the one or more other plant extracts is in the form of green tea extract.

7. The oral care composition as claimed in claim 6, in which the composition includes Melaleuca alternifolia essential oil, Mentha piperita essential oil, and concentrated green tea extract.

8. The oral care composition as claimed in claim 7, in which the potentially pathogenic oral microorganism is Prevotella intermedia and the concentration of the Heteropyxis natalensis extract is at least 3.13 mg/ml, the concentration of the concentrated green tea extract is at least 2 mg/ml, the concentration of Mentha piperita essential oil is at least 0.05% (v/v), and the concentration of Melaleuca alternifolia essential oil is at least 0.05% (v/v).

9. The oral care composition as claimed in claim 7, in which the potentially pathogenic oral microorganism is Candida albicans and the concentration of the Heteropyxis natalensis extract is at least 3.13 mg/ml, the concentration of the concentrated green tea extract is at least 4 mg/ml, the concentration of Mentha piperita essential oil is at least 0.05% (v/v), and the concentration of Melaleuca alternifolia essential oil is at least 0.01% (v/v).

10. The oral care composition as claimed in claim 7, in which the potentially pathogenic oral microorganism is Streptococcus mutans and the concentration of the Heteropyxis natalensis extract is at least 0.78 mg/ml, the concentration of the concentrated green tea extract is at least 0.78 mg/ml, the concentration of Mentha piperita essential oil is at least 0.002% (v/v), and the concentration of Melaleuca alternifolia essential oil is at least 0.0004% (v/v).

11. The oral care composition as claimed in claim 7, in which the potentially pathogenic oral microorganisms includes Streptococcus mutans, Prevotella intermedia and Candida albicans and the concentration of the Heteropyxis natalensis extract is at least 3.13 mg/ml, the concentration of the concentrated green tea extract is at least 4 mg/ml, the concentration of Mentha piperita essential oil is at least 0.05% (v/v), and the concentration of Melaleuca alternifolia essential oil is at least 0.05% (v/v).

12. Use of a Heteropyxis natalensis extract in the manufacturing of an oral care composition for the treatment of periodontal disease.

13. Use of a composition which includes Heteropyxis natalensis extract, Melaleuca alternifolia essential oils, Mentha piperita essential oils and concentrated green tea extract in the manufacturing of an oral care composition for the treatment of periodontal disease by the composition's antioxidant activity.

14. A substance or composition comprising the oral care composition as claimed in claim 1, for use in a method of treating periodontal disease.

15. An oral care composition for inhibiting the growth of potentially pathogenic oral microorganisms, said composition including Aurentiacin A or derivatives thereof.

16. Use of Aurentiacin A or derivatives thereof for inhibiting the growth of potentially pathogenic oral microorganisms.

17. A substance or composition comprising Aurentiacin A or derivatives thereof, for use in a method of treating periodontal disease.

18. A method of evaluating the attachment of microorganisms to an enamel surface of a tooth and the effect of a composition on such attachment, which includes
adding the composition to prepared enamel blocks;
adding microorganisms to the prepared enamel blocks; and
determining colonization of the microorganisms on the enamel through scanning electron microscopy (SEM).

19. The method as claimed in claim 18, in which the prepared enamel blocks are obtained by extracting teeth from humans, sterilizing the teeth, removing the crowns of the teeth and cutting the crowns into blocks.
